(19) United States
(12) Patent Application Publication
(10) Pub. No.: US 2024/0350683 A1
(43) Pub. Date: Oct. 24, 2024

(54) PREPARATION OF A PH-ADJUSTED ASCORBIC ACID SOLUTION

(71) Applicant: GE Healthcare Limited, Buckinghamshire (GB)

(72) Inventors: Graeme MCROBBIE, Chalfont St. Giles (GB); Torgrim ENGELL, Oslo (NO); Kristine WIKENE, Oslo (NO); Imtiaz Ahmed KHAN, Chalfont St. Giles (GB); Julian GRIGG, Chalfont St. Giles (GB); Alexander JACKSON, Bracknell (GB); Alan CLARKE, Oslo (NO); Jonathan Robert SHALES, Chalfont St. Giles (GB)

(21) Appl. No.: 18/569,177

(22) PCT Filed: Jun. 16, 2022
(86) PCT No.: PCT/EP2022/066481

§ 371 Date: Dec. 11, 2023

(30) Foreign Application Priority Data
Jun. 16, 2021 (GB) 2108608.7

(51) Int. Cl. A61K 51/12; A61K 47/22; C07B 59/00

(52) U.S. Cl. CPC A61K 51/121; A61K 47/22; C07B 59/004; C07B 2200/05

(57) ABSTRACT

Provided is a method of preparing an aqueous ascorbic acid solution having a pH of 2.0 to 4.0, the method comprising: providing an initial aqueous solution of ascorbic acid and a base, wherein the initial solution has a pH of 5.0 to 8.0; and combining the initial solution with a second acid to obtain an ascorbic acid solution having a pH of 2.0 to 4.0. Also provided is the use of an aqueous ascorbic acid solution having a pH of 2.0 to 4.0 prepared by a described method as a radiostabiliser of a radio-labelled compound.

### FIELD OF THE INVENTION

The present invention generally relates to a method of preparing a pH-adjusted ascorbic acid solution, the use of such a solution as a stabiliser of a radio-labelled compound and a system and a method for purifying a reaction mixture comprising a radio-labelled compound.

### BACKGROUND OF THE INVENTION

Ascorbic acid has many industrial applications. However, it is known to be chemically unstable in solution. For example, aqueous ascorbic acid solutions (in the region of pH 2) turn yellow after several hours. Pharmacopoeias recommend only short-term storage of ascorbic acid solutions, ensuring protection from light, metal ions, and elevated temperatures. This means that it is typically necessary for aqueous ascorbic acid solutions to be prepared on the same day of use.

One application of ascorbic acid is as a radiostabiliser. Radiostabilisers can be used in the manufacture of radiopharmaceuticals, pharmaceutical compounds that contain a radionuclide. Ascorbic acid in particular is a more efficient radiostabiliser at low pH, i.e. in its protonated state.

Radiostabilisers are necessary because at relatively high concentrations the radiation can trigger decomposition of the radiopharmaceutical by radiolysis. This is particularly likely during purification, when radioactivity can be concentrated on a column in a tight band. To minimise radiolysis and improve process yields and radiochemical purity (RCP), radiopharmaceuticals can be stabilised with ascorbic acid.

Automated synthesis systems are important for the production of radiopharmaceuticals. Synthesis systems of the prior art are described in WO 2007/042781 and WO 2011/097649.

Synthesis systems, such as the FASTlab® (GE Healthcare) provide for production of doses of radiopharmaceuticals for clinical applications. The FASTlab synthesizer operates a method through a device for producing a radiopharmaceutical.

Such synthesis systems/devices are used with cassettes which are customized for the specific radiopharmaceutical. Such cassettes typically include a flowpath and valves oriented along said flowpath, selectively fluidly connected to one of a number of components used for synthesizing a particular radiopharmaceutical, such as the necessary reagent vials, reaction vessels, purification cartridges, syringes, tubings, connectors and the like. The device is configured to cooperatively engage the components of the cassette so as to be able drive a source fluid with a radioisotope through the device, by operating pumps, syringes, valves, and the like, as well as controlling the provision of a motive gas (e.g. nitrogen) and the application of vacuum, to achieve a chemical synthesis process.

A fully automated, single-use, cassette type synthesis product would incorporate all necessary synthesis and purification components. A system that can be provided for the end user with all necessary reagents for the synthesis and purification, requiring minimal input from the user to provide a radiopharmaceutical ready for use, is commercially attractive. Despite being an effective radiostabiliser, there are challenges to using ascorbic acid in automated, single-use, cassette type products because it does not have a long enough shelf life to be commercially viable. It would be beneficial to provide a method enabling the production of an acidic aqueous ascorbic acid solution (in the region of pH 2-4) that overcomes the challenges presented by the short shelf life due to ascorbic acid degradation prior to use.

### SUMMARY OF THE INVENTION

In a first aspect, the present invention provides a method of preparing an aqueous ascorbic acid solution having a pH of 2.0 to 4.0, the method comprising: providing an initial aqueous solution of ascorbic acid and a base, wherein the initial solution has a pH of 5.0 to 8.0; and combining the initial solution with a second acid to obtain an ascorbic acid solution having a pH of 2.0 to 4.0.

In a second aspect, the present invention provides use of an aqueous ascorbic acid solution having a pH of 2.0 to 4.0 prepared by the method of the first aspect of the invention as a radiostabiliser of a radio-labelled compound.

In a third aspect, the present invention provides a method of stabilising a radio-labelled compound, the method comprising: preparing an aqueous ascorbic acid solution having a pH of 2.0 to 4.0 according to the method of the first aspect of the invention; and combining at least a portion of the aqueous ascorbic acid solution with a radio-labelled compound.

In a fourth aspect, the present invention provides a system for the purification of a reaction mixture comprising a radio-labelled compound, the system comprising:
(i) a flowpath; and
(ii) a plurality of valves oriented along said flowpath, wherein each of said plurality of valves is selectively fluidly connected to one of a number of components, wherein the components comprise:
   a) a composition vial for receiving a reaction mixture;
   b) a vial of an initial aqueous solution of ascorbic acid and a base, the initial aqueous solution having a pH of 5.0 to 8.0;
   c) a vial of a second acid;
   d) one or more solid phase extraction (SPE) cartridges; and
   e) one or more solvent vials.

In a fifth aspect, the present invention provides a method for stabilising a radio-labelled compound during purification of the radio-labelled compound using a system as defined in the fourth aspect of the invention, the method comprising:
(i) combining the second acid with the initial ascorbic acid solution to obtain an aqueous ascorbic acid solution having a pH of 2.0 to 4.0;
(ii) passing a reaction mixture comprising a radio-labelled compound from the composition vial into at least one of the one or more SPE cartridges;
(iii) eluting the compound to be purified from the SPE cartridge,
wherein the method further comprises
   a) mixing at least a portion of the aqueous ascorbic acid solution having a pH of 2.0 to 4.0 with the reaction mixture before step (ii);
   b) washing the one or more SPE cartridges with at least a portion of the aqueous ascorbic acid solution having a pH of 2.0 to 4.0 after step (ii) and prior to step (iii); or
   c) mixing a portion of the aqueous ascorbic acid solution having a pH of 2.0 to 4.0 with the reaction mixture before step (ii) and washing the one or more SPE cartridges with at least a portion of the aqueous ascorbic acid solution having a pH of 2.0 to 4.0 after step (ii) and prior to step (iii).

The aqueous ascorbic acid solution having a pH of 2.0 to 4.0 may be present only before and/or during a process of purification of the radiolabelled compound. The aqueous ascorbic acid solution having a pH of 2.0 to 4.0 does not form part of the purified product comprising the radiolabelled compound.

In a sixth aspect, the present invention provides a kit comprising:
a) a cassette comprising:
   (i) a flowpath; and
   (ii) a plurality of valves oriented along said flowpath, wherein each of said plurality of valves is selectively fluidly connected to one of a number of components,
b) one or more composition vials;
c) a vial of an initial aqueous solution of ascorbic acid and a base, the initial aqueous solution having a pH of 5.0 to 8.0;
d) a vial of a second acid;
e) one or more SPE cartridges; and
f) one or more solvent vials.

### FIGURES

FIG. 1 provides an example of a FASTlab cassette layout which can be used with the present invention.

FIG. 2 provides chromatograms showing the radiochemical purity of [¹⁸F] flurpiridaz prepared with (bottom) and without (top) use of ascorbic acid in the purification process.

### DETAILED DESCRIPTION OF THE INVENTION

The term ‘radiopharmaceutical’ has its conventional meaning, and refers to a radioactive compound suitable for in vivo mammalian administration for use in diagnosis or therapy. A radiopharmaceutical as referenced herein may be a PET tracer.

By the term ‘radiostabiliser’ it is meant a compound that inhibits degradation reactions, such as redox processes, by trapping highly-reactive free radicals, such as oxygen-containing free radicals arising from the radiolysis of water. Radiostabilisers protect radio-labelled compound(s) from radiolysis and therefore lower/prevent a drop in the purity of the radio-labelled compound(s) over their shelf life. Reference herein to stabilising a radio-labelled compound refers to protecting a radio-labelled compound from radiolysis.

Radiochemical purity (RCP) is determined using radio-thin layer chromatography (radio-TLC) or high-performance liquid chromatography (HPLC) and can be defined as the ratio of the (radio-labelled) drug substance peak to the total (radio-labelled) peaks in the chromatogram. If one manufactures a radiopharmaceutical with high radioactive concentration (RAC), the drop in RCP during storage is likely to be higher than at lower RAC due to more radiolysis. High radioactivity results in the drug substance destroying itself (i.e. radiolysis).

The term ‘comprising’ has its conventional meaning throughout this application and implies that the method, system, product or the like must have the components listed, but that other, unspecified components may be present in addition.

A ‘vial’ as referenced herein is a vessel that may be used to contain a liquid. A vial may be sealed using a cap, stopper or both. A vial may be made of glass or plastic and may be sealed with a plastic or rubber stopper, a metal cap, or a combination of both. Liquid contained within a vial may be extracted, for example, by means of syringing from the sealed vial (e.g. through a stopper). Vials may be formed of clear or non-clear (coloured) glass or plastic. A clear vial, which is transparent and non-coloured, allows the contents of the vial to be viewed, for example, to examine the colour of the solution. Non-clear (or coloured) vials can be used to shield the contents of a vial from light, for example in situations where this is necessary to prevent degradation by light. An ‘ampoule’ as referenced herein is a glass container sealed by melting of the top of the container to form a sealed neck, such that snapping of the neck is required to release contents of the ampoule. A vial as referenced herein is preferably not an ampoule.

An ‘inert gas’ as referenced herein may be, for example, nitrogen, argon, or a mixture thereof. The inert gas may be of European Pharmacopoeia quality.

In a first aspect, the present invention provides a method of preparing an aqueous ascorbic acid solution having a pH of 2.0 to 4.0, the method comprising: providing an initial aqueous solution of ascorbic acid and a base, wherein the initial solution has a pH of 5.0 to 8.0; and combining the initial solution with a second acid to obtain an ascorbic acid solution having a pH of 2.0 to 4.0.

It has been determined that an aqueous solution of ascorbic acid, pH adjusted by addition of a base to a pH of 5.0 to 8.0, exhibits good stability. The degradation seen for low pH ascorbic acid solutions is not observed for solutions having a pH of 5.0 to 8.0. Accordingly, this process enables a low pH ascorbic acid solution (for example of pH 2.0-4.0, preferably 2.0-3.0) to be prepared prior to use, whilst mitigating the problem of short shelf life by enabling the ascorbic acid solution to be stored in a stable pH-adjusted form. Such a method can be used, for example, in automated systems which utilise a premixed pH-adjusted ascorbic acid solution that can be used to prepare a low pH ascorbic acid solution (for example of pH 2.0-4.0, preferably 2.0-3.0) prior to use.

The pH of the initial solution may be from 5.5 to 7.5, preferably from 5.8 to 6.7, more preferably from 5.8 to 6.5, more preferably from 6.0 to 6.5.

The ascorbic acid concentration in the initial solution may be between 1 mg/ml and 100 mg/mL. The concentration may be between 10 mg/mL and 100 mg/mL. The concentration may be between 10 mg/mL and 30 mg/mL.

The initial aqueous solution may be prepared by dissolving ascorbic acid in water and adjusting the pH with a base.

The base may be a metal hydroxide, a metal carbonate, or a mixture thereof. The base may be selected from sodium hydroxide, sodium carbonate, and mixtures thereof. The base may be sodium hydroxide.

The initial aqueous ascorbic acid solution may be purged with an inert gas, such as nitrogen or argon.

It will be appreciated by a person skilled in the art that the second acid is preferably not ascorbic acid. The second acid may be a mineral acid. The mineral acid may be selected from phosphoric acid, nitric acid, sulfuric acid, hydrochloric acid, and mixtures thereof. The mineral acid may be a mineral acid that is not hydrochloric acid. The mineral acid may be phosphoric acid. The second acid may be provided in aqueous solution, for example at an acid concentration of 200-250 mg/mL.

The initial solution may be stored for a period of at least 12 hours prior to combining the initial solution with a second acid to obtain an ascorbic acid solution having a pH of 2.0 to 4.0. The initial solution may be stored for a period of at least 24 hours, at least 7 days, at least 6 months or at least a year, and preferably at least 2 years, prior to combining with the second acid.

The initial solution may be provided in a vial comprising the solution and an inert gas. The inert gas may fill the headspace of the vial. The vial may be a clear glass vial. The vial may be sealed with a stopper and/or a cap. The initial solution may be combined with the second acid using a syringe, for example by syringing the second acid into the vial.

The initial solution is combined with a second acid to obtain an ascorbic acid solution having a pH of 2.0 to 4.0. It will be appreciated that a suitable amount of second acid can be used to provide the ascorbic acid solution having a pH of 2.0 to 4.0. The method of the first aspect of the invention may be for preparing an ascorbic acid solution having a pH of 2.0 to 3.2, preferably of 2.0 to 3.0, more preferably of 2.1 to 2.6, for example 2.5. Accordingly, the initial ascorbic acid solution may be combined with a second acid to obtain an ascorbic acid solution having a pH of 2.0 to 3.2, preferably of 2.0 to 3.0, more preferably of 2.1 to 2.6, for example 2.5.

The initial aqueous ascorbic acid solution may exhibit good stability from degradation without requiring the inclusion of any additional stabilizer or preservative. The initial solution may be free of a metal-complexing (chelating) agent. For example, the solution may be free of ethylenediaminetetraacetic acid (EDTA), sodium diethyldithiocarbamate, propyl gallate, dimercaptopropanol, 8-hydroxyquinoline and amino polycarboxylic acids (such as diethylenetriaminepentaacetic acid (DTPA), N-hydroxyethylethylenediaminetriacetic acid (HEDTA)), and salts thereof. The initial solution may be free of any radio-labelled compound. The solution may consist essentially of ascorbic acid and base dissolved in water. It will be appreciated that within the solution, the ascorbic acid may exist as ascorbate, for example as sodium ascorbate if the base is sodium hydroxide or sodium carbonate.

In a second aspect, the present invention provides use of an aqueous ascorbic acid solution having a pH of 2.0 to 4.0 prepared by the method of the first aspect of the invention as a radiostabiliser of a radio-labelled compound. The use may comprise combining an aqueous ascorbic acid solution having a pH of 2.0 to 4.0 prepared according to the method of the first aspect of the invention with a radio-labelled compound before or during a process of purification of the radio-labelled compound. The purification may be SPE or HPLC purification. The use may comprise combining an aqueous ascorbic acid solution having a pH of 2.0 to 4.0 prepared according to the method of the first aspect of the invention with a radio-labelled compound to provide a radiopharmaceutical composition comprising the radio-labelled compound and ascorbic acid. The aqueous ascorbic acid solution having a pH of 2.0 to 4.0 may be present only before and/or during purification of the radio-labelled compound.

In a third aspect, the present invention provides a method of stabilising a radio-labelled compound, the method comprising: preparing an aqueous ascorbic acid solution having a pH of 2.0 to 4.0 according to the method of the first aspect of the invention; and combining at least a portion of the aqueous ascorbic acid solution with a radio-labelled compound.

The method of stabilising a radio-labelled compound may be a method of stabilising a radio-labelled compound during purification of the radio-labelled compound. The purification may be purification of a radio-labelled compound by solid phase extraction (SPE) or high-performance liquid chromatography (HPLC). Preferably, the purification may be purification by SPE. Purification may be carried out on a reaction mixture including the radio-labelled compound in a mixture with one or more impurities. Combining the aqueous ascorbic acid solution having a pH of 2.0 to 4.0 with the radio-labelled compound may occur by: a) mixing at least a portion of the aqueous ascorbic acid solution having a pH of 2.0 to 4.0 with the radio-labelled compound before loading the radio-labelled compound onto a SPE cartridge or HPLC column; b) loading the radio-labelled compound onto a SPE cartridge or HPLC column and washing the SPE cartridge or HPLC column with at least a portion of the aqueous ascorbic acid solution having a pH of 2.0 to 4.0; or c) mixing a portion of the aqueous ascorbic acid solution having a pH of 2.0 to 4.0 with the radio-labelled compound before loading the radio-labelled compound onto a SPE cartridge or HPLC column, loading the radio-labelled compound onto a SPE cartridge or HPLC column and washing the SPE cartridge or HPLC column with a portion of the aqueous ascorbic acid solution having a pH of 2.0 to 4.0. Purification of the radio-labelled compound may be carried out in an automated purification system.

In a fourth aspect, the present invention provides a system for the purification of a reaction mixture comprising a radio-labelled compound, the system comprising:
(i) a flowpath; and
(ii) a plurality of valves oriented along said flowpath, wherein each of said plurality of valves is selectively fluidly connected to one of a number of components, wherein the components comprise:
   a) a composition vial for receiving a reaction mixture;
   b) a vial of an initial aqueous solution of ascorbic acid and a base, the initial aqueous solution having a pH of 5.0 to 8.0;
   c) a vial of a second acid;
   d) one or more SPE cartridges; and
   e) one or more solvent vials.

The system of the invention has a flowpath. The flowpath is a channel that is suitable for transporting materials, particularly fluids, such as solvents and the reaction mixture. The flowpath may be configured to allow combining of the initial solution with the second acid to obtain an ascorbic acid solution having a pH of 2.0 to 4.0. The ‘reaction mixture’ may include the radio-labelled compound in a mixture with one or more impurities.

The aqueous ascorbic acid solution having a pH of 2.0 to 4.0 may be present only before and/or during purification of the radio-labelled compound.

By ‘fluidly connected’, it is meant that fluid can pass into and from the vials and (selectively) through the valves to other parts of the system. A suitable valve may be a 3-way valve having three ports and means to put any two of the three associated ports in fluid communication with each other while fluidly isolating the third port. A suitable valve may also be a stopcock valve comprising a rotatable stopcock.

The system of may comprise a cassette. The cassette houses one or more of the components of the system. The cassette may be configured to connect with any components of the system not housed therein.

The system may be joined to, or compatible with, a system or device for synthesis of a radio-labelled compound, for example the FASTlab system. A system or device for synthesis of a radio-labelled compound may provide for synthesis of a radio-labelled compound from a radio-labelled compound precursor, for example as described in WO 2011/097649. Accordingly, the system may be integrated with a system or device for the synthesis of a radio-labelled compound, such that an integrated system for synthesis and purification of a radio-labelled compound is provided. The integrated system may be fully automated.

The system may also comprise one or more of a product vial to receive eluted radio-labelled compound, a waste vial to receive eluted impurities, and a transfer line.

The vial containing the initial solution may be prepared by providing the initial aqueous solution of ascorbic acid and a base, wherein the solution has a pH of 5.0 to 8.0; degassing the solution with an inert gas; dispensing the solution into a vial; and degassing the vial headspace with an inert gas. The vial may then be sealed, for example, using a stopper and/or a cap.

The step of degassing the solution with an inert gas involves bubbling the solution through with an inert gas. Bubbling of an inert gas through the solution may be carried out while stirring the solution. This process is useful to remove undesired dissolved reactive gasses such as oxygen and carbon dioxide from the solution. The inert gas may be nitrogen, argon, or a mixture thereof. The inert gas may be nitrogen.

The step of degassing the vial headspace is useful to remove undesired reactive gasses such as oxygen and carbon dioxide from the vial headspace. The headspace of the vial refers to any space within a vial not occupied by the aqueous solution of ascorbic acid and a base. Degassing involves dispensing inert gas into the vial headspace to displace any other gases present therein. The inert gas may be nitrogen, argon, or a mixture thereof. The inert gas may be nitrogen.

The initial solution may be prepared by dissolving ascorbic acid in water and adjusting the pH with a base.

The pH of the initial solution may be from 5.5 to 7.5, preferably from 5.8 to 6.7, more preferably from 5.8 to 6.5, more preferably from 6.0 to 6.5.

The ascorbic acid concentration in the initial solution may be between 1 mg/ml and 100 mg/mL. The concentration may be between 10 mg/mL and 100 mg/mL. The concentration may be between 10 mg/ml and 30 mg/mL.

The base in the initial aqueous ascorbic acid solution may be a metal hydroxide, a metal carbonate, or a mixture thereof. The base may be selected from sodium hydroxide, sodium carbonate, and mixtures thereof. The base may be sodium hydroxide.

The initial aqueous ascorbic acid solution may exhibit good stability from degradation without requiring the inclusion of any additional stabilizer or preservative. The initial solution may be free of a metal-complexing (chelating) agent. For example, the solution may be free of ethylenediaminetetraacetic acid (EDTA), sodium diethyldithiocarbamate, propyl gallate, dimercaptopropanol, 8-hydroxyquinoline and amino polycarboxylic acids (such as diethylenetriaminepentaacetic acid (DTPA), N-hydroxyethylethylenediaminetriacetic acid (HEDTA)), and salts thereof. The initial solution may be free of any radio-labelled compound. The solution may consist essentially of ascorbic acid and base dissolved in water. It will be appreciated that within the solution, the ascorbic acid may exist as ascorbate, for example as sodium ascorbate if the base is sodium hydroxide or sodium carbonate.

The second acid is preferably not ascorbic acid. The second acid may be a mineral acid. The mineral acid may be selected from phosphoric acid, nitric acid, sulfuric acid, hydrochloric acid, and mixtures thereof. The mineral acid may be a mineral acid that is not hydrochloric acid. The mineral acid may be phosphoric acid.

The one or more solvent vials may be solvent vials containing an eluent, for example, acetonitrile in water (e.g. 40% acetonitrile), ethanol in water (e.g. 45% ethanolic solution), or another solvent system suitable for SPE elution.

In a fifth aspect, the present invention provides a method for stabilising a radio-labelled compound during purification of the radio-labelled compound using a system as defined in the fourth aspect of the invention, the method comprising:
(i) combining the second acid with the initial ascorbic acid solution to obtain an aqueous ascorbic acid solution having a pH of 2.0 to 4.0;
(ii) passing a reaction mixture comprising a radio-labelled compound from the composition vial into at least one of the one or more SPE cartridges;
(iii) eluting the compound to be purified from the SPE cartridge, wherein the method further comprises
a) mixing at least a portion of the aqueous ascorbic acid solution having a pH of 2.0 to 4.0 with the reaction mixture before step (ii);
b) washing the one or more SPE cartridges with at least a portion of the aqueous ascorbic acid solution having a pH of 2.0 to 4.0 after step (ii) and prior to step (iii); or
c) mixing a portion of the aqueous ascorbic acid solution having a pH of 2.0 to 4.0 with the reaction mixture before step (ii) and washing the one or more SPE cartridges with at least a portion of the aqueous ascorbic acid solution having a pH of 2.0 to 4.0 after step (ii) and prior to step (iii).

The aqueous ascorbic acid solution having a pH of 2.0 to 4.0 is optionally not present in the purified radio-labelled compound.

The term ‘eluting’ refers to passing a solution through a SPE cartridge with the aim to release a compound or compounds of interest that has or have been bound to the solid phase. Eluting may be carried out by passing an organic solvent through the SPE cartridge. The eluting step may additionally comprise passing an organic solvent through a transfer line for collection. The organic solvent may be an organic solvent and water mixture.

The above method may additionally comprise a step of conditioning the one or more SPE cartridges, i.e. rinsing the SPE sorbent prior to passing the reaction mixture into the one or more SPE cartridges. The conditioning step may be performed with the aqueous ascorbic acid solution having a pH of 2.0 to 4.0 after step (i) and prior to step (ii).

The above method may further comprise the step of eluting impurities. The elution of the impurities may be carried out before and/or after step (iii) above of eluting the desired product. Preferably, the elution of the impurities may be carried out before and after step (iii) above of eluting the desired product.

When the one or more SPE cartridges is washed with at least a portion of the aqueous ascorbic acid solution having a pH of 2.0 to 4.0 after step (ii) and prior to step (iii), this washing may act to elute one or more impurities (e.g. hydrophilic chemical and radiochemical impurities) and/or wash away original solvent used in the reaction mixture. One or more elution steps may be performed with a different solvent system, such as acetonitrile in water (e.g. 40% acetonitrile), to elute additional impurities and/or transfer the desired radio-labelled compound from one SPE cartridge to another SPE cartridge. The one or more elution steps may be performed after the one or more SPE cartridges is washed with at least a portion of the aqueous ascorbic acid solution having a pH of 2.0 to 4.0. Any of the one or more elution steps may be followed by a solvent exchange step, wherein the one or more SPE cartridges are washed with at least a portion of the aqueous ascorbic acid solution having a pH of 2.0 to 4.0.

Elution step (iii) may be performed with an appropriate eluent, such as an ethanolic solution (e.g. 45% ethanolic solution), in water.

Purification of the radio-labelled compound may provide the radio-labelled compound at a RCP of at least 95%, preferably at least 97%.

All features of the fourth aspect of the invention apply mutatis mutandis to the fifth aspect of the invention. The invention also provides a compound as purified according to the fifth aspect of the invention.

Various aspects of the invention refer to an aqueous ascorbic acid solution having a pH of 2.0 to 4.0. In any of these aspects the aqueous ascorbic acid solution may have a pH of 2.0 to 3.2, preferably of 2.0 to 3.0, more preferably of 2.1 to 2.6.

The radio-labelled compound as referenced in any aspect of the invention may be a radiopharmaceutical. The radio-labelled compound may be a PET tracer.

A radio-labelled compound may comprise various radio-isotopes. For example, the radio-labelled compound may be: (i) a F-18-labelled radiopharmaceutical, or a pharmaceutically acceptable salt thereof, (ii) a C-11-labelled radiopharmaceutical, or a pharmaceutically acceptable salt thereof, (iii) a C-14-labelled radiopharmaceutical, or a pharmaceutically acceptable salt thereof, (iv) a I-123-labelled radiopharmaceutical, or a pharmaceutically acceptable salt thereof; (v) a I-124-labelled radiopharmaceutical, or a pharmaceutically acceptable salt thereof; (vi) a I-125-labelled radiopharmaceutical, or a pharmaceutically acceptable salt thereof; (vii) a I-131-labelled radiopharmaceutical, or a pharmaceutically acceptable salt thereof; (viii) a Br-75-labelled radiopharmaceutical, or a pharmaceutically acceptable salt thereof; (ix) a Br-76-labelled radiopharmaceutical, or a pharmaceutically acceptable salt thereof; (x) a Br-77-labelled radiopharmaceutical, or a pharmaceutically acceptable salt thereof; (xi) a Br-78-labelled radiopharmaceutical, or a pharmaceutically acceptable salt thereof; (xii) a O-15-labelled radiopharmaceutical, or a pharmaceutically acceptable salt thereof, (xiii) a N-13-labelled radiopharmaceutical, or a pharmaceutically acceptable salt thereof, (xiv) a P-32-labelled radiopharmaceutical, or a pharmaceutically acceptable salt thereof, (xv) a Cu-62-labelled radiopharmaceutical, or a pharmaceutically acceptable salt thereof, (xvi) a Ga-67-labelled radiopharmaceutical, or a pharmaceutically acceptable salt thereof, (xvii) a Ga-68-labelled radiopharmaceutical, or a pharmaceutically acceptable salt thereof, (xviii) a Rb-82-labelled radiopharmaceutical, or a pharmaceutically acceptable salt thereof, (xix) a Sr-89-labelled radiopharmaceutical, or a pharmaceutically acceptable salt thereof, (xx) a Tc-99m-labelled radiopharmaceutical, or a pharmaceutically acceptable salt thereof, (xxi) an In-111-labelled radiopharmaceutical, or a pharmaceutically acceptable salt thereof, (xxii) a Sm-153-labelled radiopharmaceutical, or a pharmaceutically acceptable salt thereof, (xxiii) a Re-186-labelled radiopharmaceutical, or a pharmaceutically acceptable salt thereof, (xxiv) a TI-201-labelled radiopharmaceutical, or a pharmaceutically acceptable salt thereof, or combinations thereof.

The radio-labelled compound may be: (i) a F-18-labelled radiopharmaceutical, or a pharmaceutically acceptable salt thereof, (ii) a C-11-labelled radiopharmaceutical, or a pharmaceutically acceptable salt thereof, (iii) a C-14-labelled radiopharmaceutical, or a pharmaceutically acceptable salt thereof, (iv) a I-123-labelled radiopharmaceutical, or a pharmaceutically acceptable salt thereof; (v) a I-124-labelled radiopharmaceutical, or a pharmaceutically acceptable salt thereof; (vi) a I-125-labelled radiopharmaceutical, or a pharmaceutically acceptable salt thereof; (vii) a I-131-labelled radiopharmaceutical, or a pharmaceutically acceptable salt thereof; (viii) a Br-75-labelled radiopharmaceutical, or a pharmaceutically acceptable salt thereof; (ix) a Br-76-labelled radiopharmaceutical, or a pharmaceutically acceptable salt thereof; (x) a Br-77-labelled radiopharmaceutical, or a pharmaceutically acceptable salt thereof; (xi) a Br-78-labelled radiopharmaceutical, or a pharmaceutically acceptable salt thereof; (xii) a O-15-labelled radiopharmaceutical, or a pharmaceutically acceptable salt thereof, (xiii) a N-13-labelled radiopharmaceutical, or a pharmaceutically acceptable salt thereof, (xiv) a P-32-labelled radiopharmaceutical, or a pharmaceutically acceptable salt thereof, (xv) a Cu-62-labelled radiopharmaceutical, or a pharmaceutically acceptable salt thereof, (xvi) a Ga-67-labelled radiopharmaceutical, or a pharmaceutically acceptable salt thereof, (xvii) a Ga-68-labelled radiopharmaceutical, or a pharmaceutically acceptable salt thereof, or combinations thereof.

The radio-labelled compound may be a F18-labelled radiopharmaceutical, or a pharmaceutically acceptable salt thereof. Examples of such F18-labelled radiopharmaceuticals include [F-18] FDG (2-deoxy-2-[18F] fluoro-D-glucose), [F-18] FMAU (2′-deoxy-2′-[18F] fluoro-5-methyl-1-beta-D-arabinofuranosyluracil), [F-18] FMISO ([18F] fluoromisonidazole), [F-18] FHBG (9-(4-[18F]-Fluoro-3-[hydroxymethyl] butyl) guanine), [18F] FES (16a-[18F]-fluoro-17b-estradiol), [F-18] AV-45, [F-18] AV-19, [F-18] AV-1, [F-18] Flutemetamol, [F-18] Flurpiridaz, [F-18] K5, [F-18] HX4, [F-18] W372, [F-18] VM4-037, [F-18] CP18, [F-18] ML-10, [F-18] T808, [F-18] T807, 2-[F-18] fluoromethyl-L-phenylalanine, [F-18] Fluciclatide, GE-212, GE-226, or combinations thereof.

The radio-labelled compound may be a compound of Formula (I):

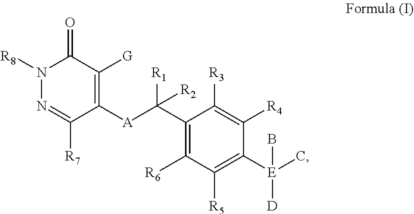

wherein A is selected from N(R⁷), S, O, C(═O), C(═O)O, NHCH₂CH₂O, a bond, or C(═O)N(R⁷);
when present, B is selected from hydrogen, alkoxyalkyl, alkyloxy, aryl, C₁-C₆ alkyl optionally substituted with an imaging moiety, heteroaryl, and an imaging moiety;
when present, C is selected from hydrogen, alkoxyalkyl, alkyloxy, aryl, C₁-C₆ alkyl optionally substituted with an imaging moiety, heteroaryl, and an imaging moiety;
D is selected from hydrogen, alkoxyalkyl, alkyloxy, aryl, C₁-C₆ alkyl optionally substituted with an imaging moiety, heteroaryl, and an imaging moiety; or
C and D, together with the atom to which they are attached, form a three- or four-membered carbocyclic ring;
G is halo or haloalkyl;
n is 0, 1, 2, or 3;
R¹, R², R³, R⁴, R⁵, R⁶ and R⁷ are independently selected from hydrogen, C₁-C₆ alkyl optionally substituted with an imaging moiety, and an imaging moiety; R⁸ is C₁-C₆ alkyl, optionally substituted with an imaging moiety; and
E is selected from a bond, carbon, and oxygen, provided that when E is a bond, B and C are absent and D is selected from aryl and heteroaryl, and provided that when E is oxygen, B and C are absent and D is selected from hydrogen, alkoxyalkyl, aryl, C₁-C₆ alkyl optionally substituted with an imaging moiety, and heteroaryl;
provided that at least one imaging moiety is present in Formula (I).

Substituent A of Formula (I) may be O. R⁸ may be tert-butyl. G may be chloro. The imaging moiety may be any radio-isotope as referenced herein, for example F-18.

The radio-labelled compound may be [F-18] Flurpiridaz, which has the following structure:

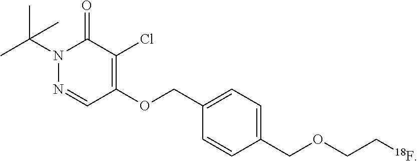

In a sixth aspect, the present invention provides a kit comprising:
a) a cassette comprising:
   (i) a flowpath; and
   (ii) a plurality of valves oriented along said flowpath, wherein each of said plurality of valves is selectively fluidly connected to one of a number of components,
b) one or more composition vials;
c) a vial of an initial aqueous solution of ascorbic acid and a base, the initial aqueous solution having a pH of 5.0 to 8.0;
d) a vial of a second acid;
e) one or more SPE cartridges; and
f) one or more solvent vials.

The initial solution and/or the second acid may be as defined for any of the preceding aspects of the invention. The flowpath may be configured to allow combining of the initial solution with the second acid to obtain an ascorbic acid solution having a pH of 2.0 to 4.0. The kit may also comprise one or more of a product vial to receive eluted radio-labelled compound, a waste vial to receive eluted impurities, and a transfer line.

The methods, systems and kit of the invention provide for a stabilised ascorbic acid solution that can be stored until ready for use, then pH adjusted in-situ at the point of use, for example as an effective radiostabiliser.

The invention is further described with reference to the following non-limiting examples.

### EXAMPLES

### Example 1: Radiosynthesis of [¹⁸F] Flurpiridaz with SPE Purification with Ascorbic Acid

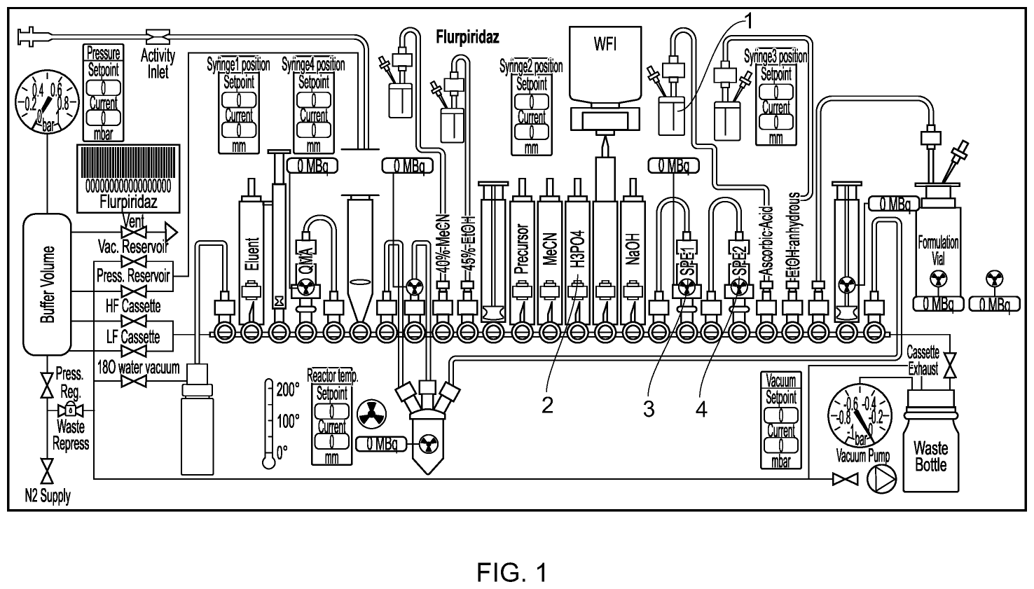

This process was carried out using FASTlab™ cassette set up as shown in FIG. 1. Components of this set up include a vial of an initial aqueous solution of ascorbic acid and a base 1, a vial of a second acid, in this example phosphoric acid 2 and two SPE cartridges 3, 4.

In the preliminary steps of the FASTlab™ process (before addition of [18F] fluoride), a solution of phosphoric acid (225 mg/ml, 3.7 mL) was added to a solution of ascorbic acid (20 mg/mL, pH 6.5, 61.5 mL). The combined solution had a pH of 2.5 and an ascorbic acid concentration of 18.9 mg/mL, and this was used in the automated FASTlab™ synthesis.

[¹⁸F] Fluoride (ca. 345 GBq) was produced using a GE Medical Systems PETtrace cyclotron with a silver target via the [¹⁸O](p,n) [¹⁸F] nuclear reaction. Total target volumes of 3.2 to 4.8 mL were used. The radiofluoride was trapped on a Waters QMA cartridge (pre-conditioned with carbonate), and the fluoride was eluted with a solution of tetrabutylammnonium hydrogen carbonate (22 mg) in water (100 μL) and acetonitrile (400 μL). Nitrogen was used to drive the solution off the QMA cartridge into the reactor vessel. The [¹⁸F] fluoride was dried for ca. 20 minutes at 110° C., including 3×0.5 mL acetonitrile azeotropic drying steps, under a steady stream of nitrogen and vacuum. The precursor (10.2 mg) in acetonitrile (1.7 mL) was added to the dried [¹⁸F] fluoride and the reaction mixture was heated at 110° C. for 3 minutes. The crude product was diluted with 2 M NaOH (2.0 mL) and aqueous ascorbic acid (3.8 mL) and left to stand for 3 minutes. The crude product was then loaded onto a tC18 SPE cartridge (Waters, product number WAT036800) and purified using the method described below.

The SPE cartridge was washed with aqueous ascorbic acid (13.4 mL) to wash away the acetonitrile, NaOH and hydrophilic chemical and radiochemical impurities. Then the SPE cartridge was washed with 40% acetonitrile (11.9 mL) to remove the hydroxy impurity. After this, the first SPE cartridge was connected in series to a second SPE cartridge (Waters, product number WAT036800) and the two were washed with further 40% acetonitrile (22.2 mL) to transfer [18F] flurpiridaz onto the second cartridge and trap the more lipophilic chemical and radiochemical impurities on the 1st SPE cartridge. The second SPE cartridge was then washed with more 40% acetonitrile (5.1 mL). The second cartridge was then washed with aqueous ascorbic acid (20.1 mL) and eluted with a 45% ethanolic solution (7 mL) to elute [18F] flurpiridaz into the product vial. The 45 mL product vial was composed of ethanol (ca. 7% v/v) and ascorbic acid (35 mg/mL).

Table 1 summarises the results from the synthesis.

TABLE 1

| Parameter | Result | |
|---|---|---|
| Non-decay corrected yield | 31% | |
| RAC | 2396 | MBq/mL |
| RCP | 98% | |
| Radioimpurity A | 1.1% | |
| Radioimpurity B | 1.1% | |
| Chemical content | 0.53 | μg/mL |
| (cyano impurity) | | |
| Chemical content | 0.13 | μg/mL |
| (non-radioactive analog of the | | |
| drug substance) | | |
| Chemical content | Not detected | |
| (chloro impurity) | | |
| Chemical content (total) | 0.66 | μg/mL |

### Example 2: Radiosynthesis of [¹⁸F] Flurpiridaz with SPE Purification without Ascorbic Acid

The same procedure as outlined in Example 1 was performed using water instead of ascorbic acid to dilute the crude product, wash the SPE cartridges, and make up the product vial.

Table 2 summarises the results from the synthesis.

TABLE 2

| Parameter | Result | |
|---|---|---|
| Non-decay corrected yield | 20% | |
| RAC | 1562 | MBq/mL |
| RCP | 84% | |
| Radioimpurity A | 3.3% | |
| Radioimpurity B | 10.1% | |
| Chemical content | Not detected | |
| (cyano impurity) | | |
| Chemical content | 0.11 | μg/mL |
| (non-radioactive analog of the | | |
| drug substance) | | |
| Chemical content | 0.12 | μg/mL |
| (chloro impurity) | | |
| Chemical content (total) | 0.37 | μg/mL |

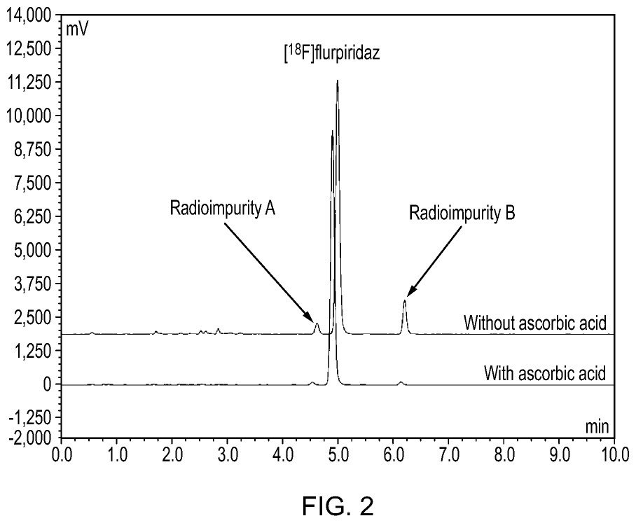

FIG. 2 shows a chromatogram of the SPE purified product with and without ascorbic acid present.

The results of examples 1 and 2 show that inclusion of ascorbic acid as part of the purification step results in a better process yield (i.e. less radiolysis of the product during purification) and a better RCP. It is desirable for RCP to be at least 95% at product release.

### Example 3: Ascorbic Acid Titration

In order to ensure that the pH of the solution obtained by combining the initial ascorbic acid solution with phosphoric acid was less than, or equal to, pH 3, it is necessary to marry the specifications of the initial ascorbic acid solution and the second acid vials. This is illustrated by the following two scenarios. The specifications of the initial ascorbic acid solution and the second acid vials, and the pH of the combined solution, are detailed in Table 3.

TABLE 3

| Parameter | High pH case | Low pH case |
|---|---|---|
| Ascorbic acid fill volume (mL) | 63 | 60 |
| Ascorbic acid concentration (mg/mL) | 21.6 | 21.4 |
| Volume of phosphoric acid added (mL) | 3.5 | 3.9 |
| Phosphoric acid concentration (mg/mL) | 220 | 240 |
| pH of ascorbic acid before addition of | 6.7 | 6.1 |
| phosphoric acid | | |
| pH of ascorbic acid after addition of | 3.0 | 2.4 |
| phosphoric acid | | |

### Example 4: Manufacturing Process for a Typical 27.3 L Batch of Ascorbic Acid (20.0 mg/mL) Vials

Vials containing an initial aqueous solution of ascorbic acid and a base suitable for use in the methods disclosed herein can be prepared using a process as outlined below.
1. Dissolve 122.85 g sodium hydroxide in 3277.15 g water for injection on a magnetic stirrer for 10 minutes.
2. Dissolve 546.0 g L-(+)-ascorbic acid in 22.5 kg water for injection on a magnetic stirrer for 20 minutes. While mixing, bubble through the solution with nitrogen gas and protect from light.
3. Add the sodium hydroxide solution to the ascorbic acid solution. Start mixing immediately after combining the solutions, or preferably while combining the solutions. Mix for 10 minutes while bubbling through the solution with nitrogen gas. The solution no longer requires protection from light.
4. Measure the pH of the solution. The pH should be 5.8-6.5.
5. Adjust the final volume of the batch by adding water for injection, q.s. to 27.3 L.
6. Mix for 15 minutes while bubbling through with nitrogen.
7. Filter the solution through a 0.2 μm filter to reduce bioburden.
8. Dispense into 100 mL Type I glass vials: degas the empty vial with nitrogen until brimful with nitrogen (typically 2 seconds), dispense ascorbic acid (20.0 mg/mL) into the vial, degas the vial headspace (typically 2 seconds), and add stopper and cap.

It will be readily understood by those persons skilled in the art that the embodiments of the inventions described herein are capable of broad utility and application. Accordingly, while the invention is described herein in detail in relation to the exemplary embodiments, it is to be understood that this disclosure is illustrative and exemplary of embodiments and is made to provide an enabling disclosure of the exemplary embodiments. The disclosure is not intended to be construed to limit the embodiments of the invention or otherwise to exclude any other such embodiments, adaptations, variations, modifications and equivalent arrangements. The scope of the invention is defined by the appended claims.

1. A method of preparing an aqueous ascorbic acid solution having a pH of 2.0 to 4.0, the method comprising:
providing an initial aqueous solution of ascorbic acid and a base, wherein the initial solution has a pH of 5.0 to 8.0; and
combining the initial solution with a second acid to obtain an ascorbic acid solution having a pH of 2.0 to 4.0.

2. The method of claim 1, wherein the initial solution has a pH of 5.8 to 6.5.

3. The method of claim 1, wherein an ascorbic acid concentration in the initial solution is between 1 mg/mL and 100 mg/mL.

4. The method of claim 1, wherein the base in the initial solution is selected from sodium hydroxide, sodium carbonate, and mixtures thereof.

5. The method of claim 4, wherein the base is sodium hydroxide.

6. The method of claim 1, wherein the second acid is a mineral acid.

7. The method of claim 6, wherein the mineral acid is selected from phosphoric acid, nitric acid, sulfuric acid, hydrochloric acid, and mixtures thereof.

8. The method of claim 7, wherein the mineral acid is phosphoric acid.

9. The method of claim 1, wherein the initial solution is stored for a period of at least 12 hours prior to combining the initial solution with a second acid to obtain an ascorbic acid solution having a pH of 2.0 to 4.0.

10. A radiostable formulation comprising the aqueous ascorbic acid solution having a pH of 2.0 to 4.0 prepared by the method of claim 1, and a radio-labelled compound.

11. A method of stabilising a radio-labelled compound, the method comprising:
preparing an aqueous ascorbic acid solution having a pH of 2.0 to 4.0 according to the method of claim 1; and
combining at least a portion of the aqueous ascorbic acid solution with a radio-labelled compound.

12. The method of claim 11, wherein the method is a method of stabilising a radio-labelled compound during purification of the radio-labelled compound.

13. The method of claim 12, wherein the purification is purification of a radio-labelled compound by solid phase extraction (SPE) or high-performance liquid chromatography (HPLC) and wherein combining the aqueous ascorbic acid solution having a pH of 2.0 to 4.0 with the radio-labelled compound occurs by:
a) mixing at least a portion of the aqueous ascorbic acid solution having a pH of 2.0 to 4.0 with the radio-labelled compound before loading the radio-labelled compound onto a SPE cartridge or HPLC column; or
b) loading the radio-labelled compound onto a SPE cartridge or HPLC column and washing the SPE cartridge or HPLC column with at least a portion of the aqueous ascorbic acid solution having a pH of 2.0 to 4.0; or
c) mixing a portion of the aqueous ascorbic acid solution having a pH of 2.0 to 4.0 with the radio-labelled compound before loading the radio-labelled compound onto a SPE cartridge or HPLC column, loading the radio-labelled compound onto a SPE cartridge or HPLC column and washing the SPE cartridge or HPLC column with a portion of the aqueous ascorbic acid solution having a pH of 2.0 to 4.0.

14. The method of claim 12, wherein purification is carried out in an automated purification system.

15. A system for purification of a reaction mixture comprising a radio-labelled compound, the system comprising:
(i) a flowpath; and
(ii) a plurality of valves oriented along said flowpath, wherein each of said plurality of valves is selectively fluidly connected to one of a number of components,
wherein the components comprise:
a) a composition vial for receiving a reaction mixture;
b) a vial of an initial aqueous solution of ascorbic acid and a base, the initial aqueous solution having a pH of 5.0 to 8.0;
c) a vial of a second acid;
d) one or more SPE cartridges; and
e) one or more solvent vials.

16. The system of claim 15, wherein the initial solution and/or the second acid are as defined in claim 2.

17. A method for stabilising a radio-labelled compound during purification of the radio-labelled compound using a system as defined in claim 15, the method comprising:
(i) combining the second acid with the initial ascorbic acid solution to obtain an aqueous ascorbic acid solution having a pH of 2.0 to 4.0;
(ii) passing a reaction mixture comprising a radio-labelled compound from the composition vial into at least one of the one or more SPE cartridges;
(iii) eluting the compound to be purified from the SPE cartridge,
wherein the method further comprises
a) mixing at least a portion of the aqueous ascorbic acid solution having a pH of 2.0 to 4.0 with the reaction mixture before step (ii);
b) washing the one or more SPE cartridges with at least a portion of the aqueous ascorbic acid solution having a pH of 2.0 to 4.0 after step (ii) and prior to step (iii); or
c) mixing a portion of the aqueous ascorbic acid solution having a pH of 2.0 to 4.0 with the reaction mixture before step (ii) and washing the one or more SPE cartridges with at least a portion of the aqueous ascorbic acid solution having a pH of 2.0 to 4.0 after step (ii) and prior to step (iii).

18. The method of claim 12, wherein the aqueous ascorbic acid solution having a pH of 2.0 to 4.0 is not present in the purified radio-labelled compound.

19. The method of claim 1, wherein the ascorbic acid solution has a pH from 2.0 to 3.2.

20. The method of claim 11, wherein the radio-labelled compound is a radiopharmaceutical.

21. The method of claim 20, wherein the radio-labelled compound comprises:
(i) a F-18-labelled radiopharmaceutical, or a pharmaceutically acceptable salt thereof,
(ii) a C-11-labelled radiopharmaceutical, or a pharmaceutically acceptable salt thereof,
(iii) a C-14-labelled radiopharmaceutical, or a pharmaceutically acceptable salt thereof,
(iv) a I-123-labelled radiopharmaceutical, or a pharmaceutically acceptable salt thereof;
(v) a I-124-labelled radiopharmaceutical, or a pharmaceutically acceptable salt thereof;
(vi) a I-125-labelled radiopharmaceutical, or a pharmaceutically acceptable salt thereof;
(vii) a I-131-labelled radiopharmaceutical, or a pharmaceutically acceptable salt thereof;
(viii) a Br-75-labelled radiopharmaceutical, or a pharmaceutically acceptable salt thereof;
(ix) a Br-76-labelled radiopharmaceutical, or a pharmaceutically acceptable salt thereof;
(x) a Br-77-labelled radiopharmaceutical, or a pharmaceutically acceptable salt thereof;
(xi) a Br-78-labelled radiopharmaceutical, or a pharmaceutically acceptable salt thereof;
(xii) a O-15-labelled radiopharmaceutical, or a pharmaceutically acceptable salt thereof,
(xiii) a N-13-labelled radiopharmaceutical, or a pharmaceutically acceptable salt thereof,
(xiv) a P-32-labelled radiopharmaceutical, or a pharmaceutically acceptable salt thereof,
(xv) a Cu-62-labelled radiopharmaceutical, or a pharmaceutically acceptable salt thereof,
(xvi) a Ga-67-labelled radiopharmaceutical, or a pharmaceutically acceptable salt thereof,
(xvii) a Ga-68-labelled radiopharmaceutical, or a pharmaceutically acceptable salt thereof,
(xviii) a Rb-82-labelled radiopharmaceutical, or a pharmaceutically acceptable salt thereof,
(xix) a Sr-89-labelled radiopharmaceutical, or a pharmaceutically acceptable salt thereof,
(xx) a Tc-99m-labelled radiopharmaceutical, or a pharmaceutically acceptable salt thereof,
(xxi) an In-111-labelled radiopharmaceutical, or a pharmaceutically acceptable salt thereof,
(xxii) a Sm-153-labelled radiopharmaceutical, or a pharmaceutically acceptable salt thereof,
(xxiii) a Re-186-labelled radiopharmaceutical, or a pharmaceutically acceptable salt thereof,
(xxiv) a Tl-201-labelled radiopharmaceutical, or a pharmaceutically acceptable salt thereof,
or combinations thereof.

22. The method of claim 21, wherein the radio-labelled compound comprises a F18-labelled radiopharmaceutical, or a pharmaceutically acceptable salt thereof.

23. The method of claim 22, wherein the radio-labelled compound comprises [F-18] FDG (2-deoxy-2-[18F] fluoro-D-glucose), [F-18] FMAU (2′-deoxy-2′-[18F] fluoro-5-methyl-1-beta-D-arabinofuranosyluracil), [F-18] FMISO ([18F] fluoromisonidazole), [F-18] FHBG (9-(4-[18F]-Fluoro-3-[hydroxymethyl] butyl) guanine), [18F] FES (16a-[18F]-fluoro-17b-estradiol) [F-18] AV-45, [F-18] AV-19, [F-18] AV-1, [F-18] Flutemetamol, [F-18] Flurpiridaz, [F-18] K5, [F-18] HX4, [F-18] W372, [F-18] VM4-037, [F-18] CP18, [F-18] ML-10, [F-18] T808, [F-18] T807, 2-[F-18] fluoromethyl-L-phenylalanine, [F-18] Fluciclatide, GE-212, GE-226, or combinations thereof.

24. The method of claim 23, wherein the radio-labelled compound comprises [F-18] Flurpiridaz:

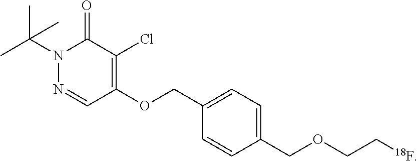

25. A kit comprising:
a) a cassette comprising:
(i) a flowpath; and
(ii) a plurality of valves oriented along said flowpath, wherein each of said plurality of valves is selectively fluidly connected to one of a number of components,
b) one or more composition vials;
c) a vial of an initial aqueous solution of ascorbic acid and a base, the initial aqueous solution having a pH of 5.0 to 8.0;
d) a vial of a second acid;
e) one or more SPE cartridges; and
f) one or more solvent vials.
